(19) United States
(12) Patent Application Publication
(10) Pub. No.: US 2023/0190791 A1
(43) Pub. Date: Jun. 22, 2023

(54) Preparation of a Brain Targeted Artificial Nano-Enzyme and Application

(71) Applicant: University of South Carolina, Columbia, SC (US)

(72) Inventors: Peisheng Xu, Chapin, SC (US); Haijun Liu, Columbia, SC (US)

(73) Assignee: University of South Carolina, Columbia, SC (US)

(21) Appl. No.: 17/971,694

(22) Filed: Oct. 24, 2022

Related U.S. Application Data
(60) Provisional application No. 63/292,528, filed on Dec. 22, 2021.

(51) Int. Cl. A61K 33/244; A61P 25/28; A61K 9/14

(52) U.S. Cl. CPC A61K 33/244; A61P 25/28; A61K 9/14

(57) ABSTRACT

Described herein are methods and systems for the preparation of a brain targeted cerium oxide nanop article (CeNP) and its application in treating central neuronal system diseases. The brain targeted CeNP (T-CeNP) can effectively pass the blood brain barrier and specifically target brain tissue and exhibit anti-inflammatory and anti-oxidant effects.

### STATEMENT REGARDING FEDERALLY SPONSORED RESEARCH

This disclosure was made with government support under NIH R01AG054839. The government has certain rights in the disclosure.

### TECHNICAL FIELD

The subject matter disclosed herein is generally directed to the preparation of a brain targeted cerium oxide nanop article (CeNP) and its application in treating central neuronal system diseases. The brain targeted CeNP (T-CeNP) can effectively pass the blood brain barrier and specifically target brain tissue and exhibit anti-inflammatory and anti-oxidant effects.

### BACKGROUND

Chronic neuroinflammation is caused by the activation of microglia, which is the major cause of central neuronal system (CNS) diseases, including Alzheimer's disease, Multiple Sclerosis, brain tumors, glioblastoma, neuroblastoma, Parkinson's disease, Epilepsy, neonatal hypoxic-ischemic, stroke, Amyotrophic Lateral Sclerosis, Huntington's disease, spinal cord injury, brain injury, post-traumatic stress disorder, and frontotemporal dementia.

Accordingly, it is an object of the present disclosure to provide novel methods, systems, and treatment methods for central neuronal system diseases. The current disclosure provides a nanoparticle which can efficiently pass the blood brain barrier (BBB) and be targeted to brain tissue. The resulting nanocarrier has anti-oxidant and anti-inflammatory effect and can be applied for the treatment of Alzheimer's disease, Multiple Sclerosis, brain tumors, glioblastoma, neuroblastoma, Parkinson's disease, Epilepsy, neonatal hypoxic-ischemic, stroke, Amyotrophic Lateral Sclerosis, Huntington's disease, spinal cord injury, brain injury, post-traumatic stress disorder, frontotemporal dementia, etc.

Citation or identification of any document in this application is not an admission that such a document is available as prior art to the present disclosure.

### SUMMARY

The above objectives are accomplished according to the present disclosure by providing in one embodiment, a method for preparation of a targeted cerium oxide nanoparticle (CeNP). The method may include preparing poly(lactide-co-glycolide)-b-poly(ethylene glycol)-maleimide (PLGA-PEG-Mal), affixing at least one alkanethiol to at least one cerium oxide nanop article; and constructing at least one nanocluster of T-CeNP via: mixing PLGA and PLGA-PEG-Mal with the at least one cerium oxide nanoparticle; and forming a solution of the above and adding at least one receptor for advanced glycation endproducts. Further, the at least one alkanethiol may comprise 1-octanethiol. Still further, an effective amount of the targeted cerium oxide nanoparticle may be introduced to at least one neuronal cell for treatment of a central neuronal system disease. Still yet, the central neuronal system disease may include Alzheimer's disease, Multiple sclerosis, Brain tumor, glioblastoma, neuroblastoma, Parkinson's disease, Epilepsy, neonatal hypoxic-ischemic, stroke, Amyotrophic lateral sclerosis, Huntington's disease, Spinal cord injury, brain injury, post-traumatic stress disorder, and/or frontotemporal dementia. Even further, the method may include an effective amount of the cerium oxide nanoparticle to at least one neuronal cell to provide antioxidant or anti-inflammatory effects to the at least one neuronal cell. Moreover, the targeted cerium oxide nanoparticle may be introduced to a subject and penetrates the blood brain barrier of the subject. Still yet further, the at least one cerium oxide nanoparticle may range in size from 2-10 nanometers. Further again, the Poly (lactic-co-glycolic acid) nano-matrix may range in size from 50-300 nanometers.

In a further aspect of the disclosure, a hybrid nanoparticle comprising a Poly (lactic-co-glycolic acid) nano-matrix and at least one cerium oxide nanoparticle is provided. Further, the at least one cerium oxide nanoparticle may be encapsulated in the Poly (lactic-co-glycolic acid) nano-matrix by hydrophobic interaction. Still yet further, the at least one cerium oxide nanoparticle may range in size from 2-10 nanometers. Even further yet, the Poly (lactic-co-glycolic acid) nano-matrix may range in size from 50-300 nanometers. Again further, the at least one cerium oxide nanoparticle may exhibit antioxidant and/or anti-inflammatory effects. Further yet, a targeting ligand may be bonded to the hybrid nanoparticle. Moreover still, the hybrid nanoparticle may be used in an effective amount for treatment of a central neuronal system disease. Still further, the central neuronal disease comprises Alzheimer's disease, Multiple sclerosis, Brain tumor, glioblastoma, neuroblastoma, Parkinson's disease, Epilepsy, neonatal hypoxic-ischemic, stroke, Amyotrophic lateral sclerosis, Huntington's disease, Spinal cord injury, brain injury, post-traumatic stress disorder, and/or frontotemporal dementia.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A shows at (a) a schematic fabrication of T-CeNP.

FIG. 1B shows at: (b) TEM images of T-CeNP; and (c) DLS size of T-CeNP and N-CeNP.

FIG. 1C shows at (d) catalase mimetic activity; and (e) SOD mimetic activity of T-CeNP and free CeNP by referencing to catalase and SOD.

FIG. 2A shows cell viability of N2a cells measured by MTT assay.

FIG. 2B shows cell viability of N2a cells measured by live/dead staining.

FIG. 3A shows at (a) cell viability of BV-2 cells measured by MTT assay after incubation with different concentration of T-CeNP for 24 h; and at (b) fluorescence images of intracellular ROS in N2a cells.

FIG. 3B shows quantitative analysis of secretive TNF-α in BV-2 by a using TNF-α ELIAS kit after following treating with various nanoparticles at (c) Aβ oligomers or (d) LPS.

FIG. 4A shows at (a) a schematic formation of Aβ oligomers, nanocomposites of T-CeNP and Aβ oligomers and their internalization in BV-2 cells; and at (b) TEM images of Aβ oligomers and nanocomposites of various nanoparticles and Aβ oligomers after 24 h of incubation.

FIG. 4B shows at (c) distribution of various Aβ oligomers in BV-2 cells.

FIG. 5A shows co-location of Aβ oligomers and lysosome in BV-2 cells.

FIG. 5B shows uptake of various nanoparticles and their aggregation with Aβ by BV-2 cells.

FIG. 6A shows at (a) confocal laser scanning microscopy images of hCMEC/D3 cells incubated with different nanoparticles for 2 and 4 h; and at (b) confocal laser scanning microscopy images of hCMEC/D3 cells pretreated with conditioned medium from BV-2 cells or LPS treated BV-2 cells, incubated with different nanop articles for 4 h.

FIG. 6B shows at (c) a schematic of in vitro BBB model and quantitative analysis of penetration efficiency of various nanoparticles through the BBB model; and (d) a schematic of in vitro BBB model and quantitative analysis of the influence of pretreatment with conditioned medium from BV-2 cells or LPS treated BV-2 cells on the penetration efficiency of various nanoparticles through the BBB model.

FIG. 7A shows in vivo and ex vivo images of T-CeNP distribution in AD mice.

FIG. 7B shows in vivo and ex vivo images of T-CeNP distribution in normal mice.

FIG. 8A shows at: (a) a time schedule of drug administration and therapeutic evaluation; (b) a photograph of nest-construction; and at (c) corresponding nest-constructing scores in different groups.

FIG. 8B shows graphs of: (e) escape latency; (f) number of times crossing the escape platform; and (g) percentage of time spent in the quadrant where the escape platform is located.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of the pharmaceutical formulation thereof calculated to produce the desired response or responses in association with its administration.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### KITS

Any of the nanop articles and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of the nanoparticle and/or pharmaceutical formulation thereof such as any of the methods described in greater detail elsewhere herein.

Chronic neuroinflammation is caused by the activation of microglia, which is the major cause of central neuronal system (CNS) diseases, including Alzheimer's disease (AD), Multiple Sclerosis, brain tumors, glioblastoma, neuroblastoma, Parkinson's disease, Epilepsy, neonatal hypoxic-ischemic, stroke, Amyotrophic Lateral Sclerosis, Huntington's disease, spinal cord injury, brain injury, post-traumatic stress disorder, frontotemporal dementia, etc.

Cerium oxide nanoparticle (CeNP), a well-known metal catalyst, shows an outstanding biomedical potential attributing to its antioxidant properties. Cerium (III, Ce³⁺) and cerium (IV, CO⁴⁺) oxidation states coexist on the surface of CeNP as a redox couple, exhibiting superoxide dismutase (SOD) and catalase (CAT) mimicking activities, respectively, which scavenges noxious intracellular reactive oxygen species (ROS). Moreover, these catalytic capabilities can be regenerated through a redox cycling mechanism. Therefore, CeNP receives a lot of attention as a promising antioxidant therapeutic agent for various oxidative stress associated diseases, such as chronic inflammation and neurodegeneration.

### Synthesis of Poly(lactide-co-glycolide)-b-poly(ethylene glycol)-maleimide (PLGA-PEG-Mal)

PLGA-PEG-Mal was prepared as known to those of skill in the art. In brief, 500 mg of carboxylic acid-terminated Poly(lactide-co-glycolide) (PLGA) polymer (0.67 dL/g, 50:50 ratio; MW: 38-52 Kd) was dissolved in 10 mL dimethylformamide (DMF), followed by adding 71.6 mg 1-ethyl-3-(−3-dimethylaminopropyl) carbodiimide hydrochloride (EDC) and 28.75 mg N-hydroxysuccinimide (NHS) and stirred for 2 h. After that, 42.5 mg NH2-PEG₃₄₀₀-Mal was added to the reaction solution, and the mixture solution was further stirred overnight, followed by dialysis with a membrane (MWCO: 10,000) in DMF and methanol sequentially and vacuum drying. The structure of PLGA-PEG-Mal was confirmed by ¹H MNR.

Preparation of T-CeNP

Firstly, the cerium oxide nanoparticle (Sigma-Aldrich Chemical Co., MO, USA) was decorated with 1-octanethiol to increase its hydrophobicity of the cerium oxide nanop article. Cerium oxide lyophilized power (500 mg) was dissolved in 10 mL mixed solvent of methanol and chloroform (1:3, v/v), and 200 μL of 1-octanethiol was added dropwise under stirring. After 24 h of stirring in dark, the mixture solution was processed by dialysis in methanol and chloroform (1:3, v/v) with a membrane bag (MWCO: 1000). The 1-octanethiol decorated Cerium oxide (CeNP) was stored at −20° C. for further use.

The nanocluster of T-CeNP was constructed by an emulsion solvent evaporation method. In brief, 100 mg of PLGA and 10 mg of PLGA-PEG-Mal were dissolved in 2 mL of chloroform, followed by addition of 0.5 mL of CeNP solution (10 mg/ml). The solution was added dropwise into 10 mL of 5% poly(vinyl alcohol) (PVA) solution under slight vortex. Then the mixed solution was emulsified by sonication using a sonicator (ULTRASONIC PROCESSOR XL, Misonix, N.Y., USA) at 70% Pulse duty cycle on ice for 15 min. The emulsion was poured into 20 ml of 0.5% PVA solution under stirring, and the organic solvent was evaporated by stirring under an atmospheric pressure at room temperature overnight. The nanoparticles were collected by centrifugation at 13,000 rcf for 20 min, washed twice with water and redispersed in phosphate-buffered saline (PBS). After that, 0.3 mg of Receptor for advanced glycation endproducts (RAGE) peptide in 0.5 mL of dimethylsulfoxide (DMSO) was added dropwise into the solution, stirred for overnight and purified by centrifugation to get the RAGE decorated CeNP (T-CeNP). The non-targeted CeNP (N-CeNP) was constructed similar as T-CeNP excepting replacing RAGE peptide with 2-mercaptoethanol. The targeted plain nanoparticles (T-PLGA) and non-targeted plain nanoparticles (N-PLGA) were prepared similar as T-CeNP and N-CeNP, respectively, excepting no CeNP was loaded.

Characterization of T-CeNP

The morphologies of the prepared nanoparticles were characterized and observed by transmission electron microscope (Hitachi HT7800 TEM, Hitachi High Technologies, Tokyo, Japan). The hydrodynamic sizes and zeta potentials of the nanoparticles were measured by Nano ZS Zetasizer (Malvern Instruments, UK).

The catalase activities of free CeNP and T-CeNP were evaluated based on the reaction of ammonium metavanadate with H₂O₂. It was established that H₂O₂ can easily reduce vanadium (V) to vanadium (III) under acidic conditions, and concurrently produce a red orange peroxovanadium complex, which had the largest absorbance peak at the wavelength of 452 nm. In brief, the nanoparticles were added to PBS contained 10 mM H₂O₂ and incubated for 10 min. Subsequently, ammonium metavanadate (0.01 M) in sulfuric acid (0.5 M) was added to the reaction solution to determine the remaining H₂O₂ through colorimetric assay. Catalase was used as standard reagent.

The SOD mimetic activity of the nanoparticles was measured using Superoxide Dismutase Assay Kit (Cayman Chemical, Ann Arbor, Mich.) according to the manufacturer's manual, SOD was used as standard reagent.

Cell Culture

Neuro-2a (N2a), SH-SY5Y and murine BV-2 microglia cell (BV-2) were obtained from American Type Culture Collection (ATCC, Manassas, Va., USA) and were cultured in Dulbecco's Modified Eagle's Medium (DMEM) containing 10% of fetal bovine serum (FBS, Gibco), 100 U/mL of penicillin and 100 mg/mL of streptomycin under a humidified atmosphere of 5% CO₂ at 37° C. The hCMEC/D3 were cultured in EndoGRO™-MV Complete Media Kit (Cat. No. SCME004) supplemented with 1 ng/mL FGF-2 (Cat. No. GF003) under a humidified atmosphere of 5% CO₂ at 37° C. All cell lines were subcultured when they reached 90% confluence, and the culture medium was replaced with fresh one every 2-3 days.

Preparation of Aβ₁₋₄₂ Monomer

The monomer, an oligomer of Aβ₁₋₄₂, was prepared. Briefly, the Aβ₁₋₄₂ peptide was dissolved in 1,1,1,3,3,3-hexafluoroisopropanol (HFIP) at the concentration of 5 mg/ml, and was bath sonicated for 15 min and incubated with shaking at room temperature for another 2 h. Subsequently, the solution was divided into aliquots in microcentrifuge tube followed by evaporation of HFIP in a vacuum oven and stored at −20° C. for further using. The aliquot was resuspended in DMSO at the concentration of 5 mM and was bath sonicated for 10 min to obtain the Aβ₁₋₄₂ monomer. The Aβ₁₋₄₂ monomer solution (5 mM) was diluted to 100 μM with deionized water and incubated at room temperature for 24 h to obtain the Aβ₁₋₄₂ oligomer.

Cell Viability Assay

MTT assay was carried out to investigate the protective effect of T-CeNP on ROS or Aβ₁₋₄₂ oligomer induced cytotoxicity to N2a cells. In brief, N2a cells were seeded in a 96-well plate (10,000 cells/well) and cultured for 24 h. Then the medium was replaced with a fresh one containing T-CeNP at different concentrations and incubated for another 12 h. After that, the medium was substituted with fresh one containing 100 μM of H₂O₂ or 10 μM of Aβ₁₋₄₂ oligomer and incubated for 24 h. Subsequently, 10 μl of MTT stock solution in PBS (5 mg/ml) was added to each well and incubated for another 4 hours, followed by discarding the medium and adding of 100 μl of DMSO. The optical density (OD) was measured at the wavelength of 570 nm, and the cell viability was calculated using following formula: OD_{X}/OD_{C}*100%, wherein OD_{C} was the OD value of control group and OD_{X} represented the experimental one. Live/dead cell staining assay was further carried out to visualize the protective effect of T-CeNP. N2a cells was processed as aforementioned MTT assay. After treatment with Aβ₁₋₄₂ oligomer for 24 h, cells were incubated with calcein-AM and EthD-1 in no FBS DMEM medium for 15 min at 37° C. with 5% CO₂ and imaged the live and dead cells according to the manufacturer's instructions using a fluorescence microscope (Evos™ FL, ThermoFisher Scientific, MA, USA).

ROS Measurement

BV-2 cells were cultured in 35 mm glass bottom dishes at the density of 25,000/well and incubated for 24 h. After that, the medium was replaced with a fresh complete medium containing 100 μM of different kind of nanoparticles and incubated for 12 h. After that, the medium was discarded, and the cells were washed with PBS for 3 time, followed by culture in complete medium containing 100 μM of H₂O₂ for another 12 h. After that, intracellular ROS was measured with 2′,7′-dichlorofluorescein diacetate (DCFH-DA), which could transform to the fluorescent product DCF in the present of esterase and ROS, indicating the intracellular oxidative stress. In brief, the culture medium was removed, and the cells were washed 3 times with PBS and incubated in no FBS medium containing 10 μl of DCFH-DA for 30 min at 37° C. After 3 times wash with PBS, the fluorescence images were taken using a fluorescence microscope (Evos™ FL, ThermoFisher Scientific, MA, USA) with Green Fluorescent Protein filter.

Enzyme-Linked Immunosorbent Assay (ELISA) of TNF-α

BV-2 cells were cultured in 6-well plates at a density of 200,000 cells/well and incubated for 24 h. Then cells were treated with 100 μM of different kind of nanoparticles for 12 h, followed by washing 3 times with PBS and incubating with 100 μM of H₂O₂ or 5 μM of Aβ₁₋₄₂ oligomer in complete medium for 24 h. After that, the medium was collected and centrifuged at 1,500 rcf for 10 min to remove cell debris. The TNF-α content in the medium was measured by Human/Mouse TNF-α ELIAS kit (Invitrogen, USA) along with the manufacturer's instructions. The total protein in cells was quantified using BCA assay.

Co-assembly and Aggregation of Aβ₁₋₄₂ and Nanoparticles

To investigate the interaction between Aβ₁₋₄₂ and nanoparticles, they were co-incubated for different times, and the morphologies of nanoparticles and Aβ₁₋₄₂ were observed through TEM. Aβ₁₋₄₂ monomer solution and nanoparticles solution were mixed sufficiently at the final concentration of 5 μM and 100 μM, respectively, and incubated at RT for different times. At special time, 10 μl of the mixed solution was dropped onto a copper mesh, dried and view the morphology by TEM (Hitachi HT7800 TEM, Hitachi High Technologies, Tokyo, Japan).

Cellular Uptake and Location of T-CeNP in BV-2 Cells

To investigate the cellular uptake and location of Aβ₁₋₄₂ oligomer and the aggregate of Aβ₁₋₄₂ and nanoparticles in BV-2 cells, Aβ₁₋₄₂ was firstly labeled with fluorescein isothiocyanate (FITC). 2 mg of Aβ₁₋₄₂ and 400 μg of FITC were dissolved in DMSO and stirred overnight under dark, followed by dialysis in DMSO with a membrane bag (MWCO:1000). The FITC labeled Aβ₁₋₄₂ monomer was prepared as the preparation of Aβ₁₋₄₂ monomer.

BV-2 cells were cultured in 35 mm glass bottom dishes at a density 4×10⁴ cells/well. After 24 h of incubation, the medium was replaced with a fresh complete medium containing 0.8 μM of Aβ₁₋₄₂ oligomer or the aggregation of Aβ₁₋₄₂ and different nanoparticles. After 4 h of incubation, the cells were washed 3 times with PBS and fixed with 4% paraformaldehyde for 15 min. The nuclei were stained with 10 μg/ml of Hoechst 33254 for 10 min at room time. The uptake and location of Aβ₁₋₄₂ oligomer were observed with a Carl Zeiss LSM700 confocal microscope.

To perform intracellular tracking of Aβ₁₋₄₂ oligomer in BV-2 cells, BV-2 cells were treated with Aβ₁₋₄₂ oligomer or the aggregation of Aβ₁₋₄₂ and different nanoparticles for 3 h as aforementioned cellular uptake, then the medium was removed and the cells were stained with Lysotracker Red DND-99 (Invitrogen, USA) according to the manufacturer's instructions. Then, the cells were washed three times with PBS, fixed with 4% paraformaldehyde for 15 min. Hoechst 33254 was used to stain nuclei. The co-location of Aβ₁₋₄₂ oligomer and lysosome were characterized by the Carl Zeiss LSM700 confocal microscope.

To investigate the internalization of nanop articles and their aggregation with Aβ₁₋₄₂ oligomer in BV-2 cells, Nile red was loaded in nanoparticles as a fluorescence probe. BV-2 cells were incubated with different kind of Nile red load nanoparticles and their aggregation with Aβ₁₋₄₂ at a Nile red concentration of 0.2 μg/ml for 4 h, then the cells were washed three times with PBS, fixed with 4% paraformaldehyde for 15 min. Hoechst 33254 was used to stain nuclei. The uptake of different nanoparticles was characterized by the Carl Zeiss LSM700 confocal microscope.

Cellular Uptake in hCMEC/D3

hCMEC/D3 were seeded in 35 mm glass bottom dishes at a density 4×10⁴ cells/well. After 24 h of culture, the medium was replaced with a fresh complete medium containing Nile red loaded T-CeNP or N-CeNP at a Nile red concentration of 0.1 μg/ml and incubated for 2 h and 4 h. Then, the cells were washed three times with PBS, fixed with 4% paraformaldehyde for 15 min. Hoechst 33254 was used to stain nuclei. The uptake of nanoparticles in hCMEC/D3 was characterized by the Carl Zeiss LSM700 confocal microscope.

To investigate the influence of inflammatory on the internalization of nanoparticle into hCMEC/D3 cells, hCMEC/D3 cells were treated with conditioned medium from BV-2 cells pretreated with lipopolysaccharides (LPS) or not for 24 h prior to uptake assay as aforementioned method.

In Vitro BBB Penetration

The BBB model was constructed as known to those of skill in the art. hCMEC/D3 cells were seed on a polycarbonate 24-well Transwell membrane of 1 μm mean pore size at a density of 10,000 cells/well. The transendothelial electrical resistance (TEER) of cell monolayers was monitored every day by using an epithelial voltohmmeter (Millicell-RES, Millipore, USA). The BBB model could be used when the TER was above 180 Ω/cm², to estimate BBB penetrating ability and efficiency of the nanoparticles. Fresh culture medium (200 μL) containing Nile red loaded T-CeNP or N-CeNP at the Nile red concentration of 0.2 μg/mL was added into the upper chamber, 600 μL of fresh complete medium was added into the lower chamber. At predesigned time points, the medium in the lower chamber was collected and replaced with fresh one. At the end of the assay, the penetrated nanoparticles in the collected medium were quantitatively measure by fluorimeter to calculate the accumulative penetrating efficiency of the nanoparticles. The accumulative penetrating efficiency in the Transwell membrane without cell monolayers was used as positive control.

To investigate the influence of inflammatory condition on the BBB penetrating efficiency of the nanop articles, the cell monolayers on the Transwell membrane were treated for 24 h with conditioned medium from BV-2 cells pretreated with LPS before performing BBB crossing assay as aforementioned method.

To further investigate whether the penetrated nanoparticles could be internalized by neuronal cells or BV-2, the BBB penetrating assay was carried out in another Transwell plate, whose lower chamber was precultured with SH-SY5Y or BV-2 cells. After 4 h of penetration assay, the upper chamber was removed, and SH-SY5Y cells were further incubated in the lower chamber for 0 and 8 hours, followed by characterizing the internalized nanoparticles by the Carl Zeiss LSM700 confocal microscope visualizing.

In Vivo Distribution of Nanoparticles in AD Mice

To investigate the in vivo brain targeting and distribution of nanoparticles in AD mice or normal mice, Cy5 loaded T-CeNP and N-CeNP were injected intravenously to 6 months old 5XFAD mice or normal mice at a Cy5 dose of 0.5 mg/kg. At predesigned time points, the mice were anesthetized and imaged using an IVIS Lumina III live animal imaging system (PerkinElmer Inc., Waltham, USA) with the filter setting of excitation: 630 nm; emission: 650-670 nm. Following the in vivo imaging, the mice were sacrificed, and the major organs (brain, heart, liver, spleen, lung and kidney) were collected for ex vivo imaging to study nanoparticle tissue distribution.

Drug Treatment of AD Mice

AD transgenic mice (5XFAD) at the age of 6 months old were randomly divided into three groups (n=7) and intravenously injected with saline, T-CeNP (1 mg/kg) and N-CeNP (1 mg/kg) every 3 days for 2 months. C57BL/6 mice (WT-like littermates) given with saline were used as Sham group.

Nest Construction Assay

The assessment of nest construction capacity was performed once a week in the duration of treatment. In brief, each mouse was caged and housed individually, approximately 1 hour before the dark phase, one pressed cotton square (5×5 cm, Nestlets, Ancare, Bellmor, USA) was placed in the home cage. Next morning, the nest was photographed and scored as follows: 1=not noticeably touched, 2=partially torn up, 3=mostly shredded but often no identifiable site, 4=identifiable but flat, 5=perfect or nearly.

Morris Water Maze Test (MWM)

To evaluate the spatial learning and memory ability of mice following different treatments, the MWM was carried. The test was performed in a circular water pool (diameter: 120 cm, height: 50 cm), which was divided into four quadrants, and an adjustable platform (diameter: 9 cm) submerged 1 cm below the surface of water in the center of one quadrant. Four different symbols (pentagram, square, triangle, and circle) were fixed on the wall of each quadrant as visible cues. The temperature of water was keep at 22±1° C. and nonfat milk power was added into the water to avoid animal tracking. In the training section, each mouse received 4 times training (inlet from each quadrant) daily to find the hidden platform within 20-30 min interval for five consecutive days. The mice were put into the pool with their heads facing the wall along the border. The cutoff time for the latency to the platform was 60 s. The time mice found the platform was recorded. If a mouse failed to reach the platform within 60 s, it was guided to the platform and keep there for 15 sec. The swimming route and the latency was recorded using an EthoVision XT® 15 tracking system (Noldus, Wageningen, The Netherlands). On the sixth day, the platform was removed and the probe trails were performed. Each mouse was placed into the pool from opposite quadrants of the targeted one, and allowed it to swim freely for 60 s. The percent of time spent in targeted quadrant and the number of targeted platform crossing were record.

Immunostaining

At the end of the behavior experiments, all mice were anesthetized by 2% isoflurane and followed by perfusion with 60 mL of saline and 60 mL of 4% paraformaldehyde solution in PBS. The brains were fixed in PBS solution containing 4% paraformaldehyde for 20 h at 4° C. and dehydrated in 30% sucrose PBS solution for 72 hours, followed by sectioning into 15 μm slices using a freezing cryostat (Leica, Wetzlar, Germany). For Immunostaining, the tissue slices were blocked in 5% BAS and followed by staining with anti-NeuN antibody (1:100; Abcam, Cambridge, Mass.), anti-GFAP antibody (1:100; Abcam, Cambridge, Mass.), anti-Iba 1 antibody (1:100; Abcam, Cambridge, Mass.), anti-TNF-α antibody (1:50; Invitrogen, Carlsbad, Calif., USA), anti-CD31 antibody (1:1000; Abcam, Cambridge, Mass.), anti-RAGE antibody (1:100; Proteintech Group, Chicago, Ill.) and anti-8OHG antibody (1:250; abcam) in 5% BSA overnight at 4° C., and then detected by appropriate secondary antibodies.

Thioflavin S Staining

Thioflavin S (1%) in H₂O was freshly prepared and filtered. The brain sections were warmed to room temperature and washed 3 times with PBS, and then were stained with thioflavin S for 10 min at room temperature. After that, the section was sequentially clipped in 75% ethanol, 95% ethanol, 100% ethanol, 95% ethanol, 75% ethanol and water for 1 min. The fluorescence images were photographed by using a fluorescence microscope (Evos™ FL, ThermoFisher Scientific, MA, USA) with the Green Fluorescent Protein filter to detect the Aβ plaques in the brain tissue.

Results

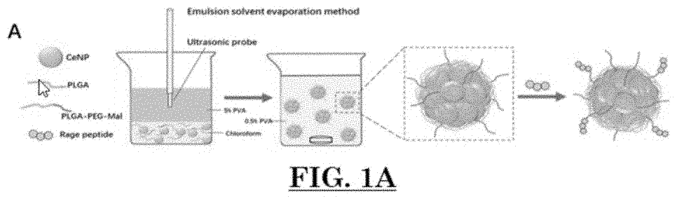

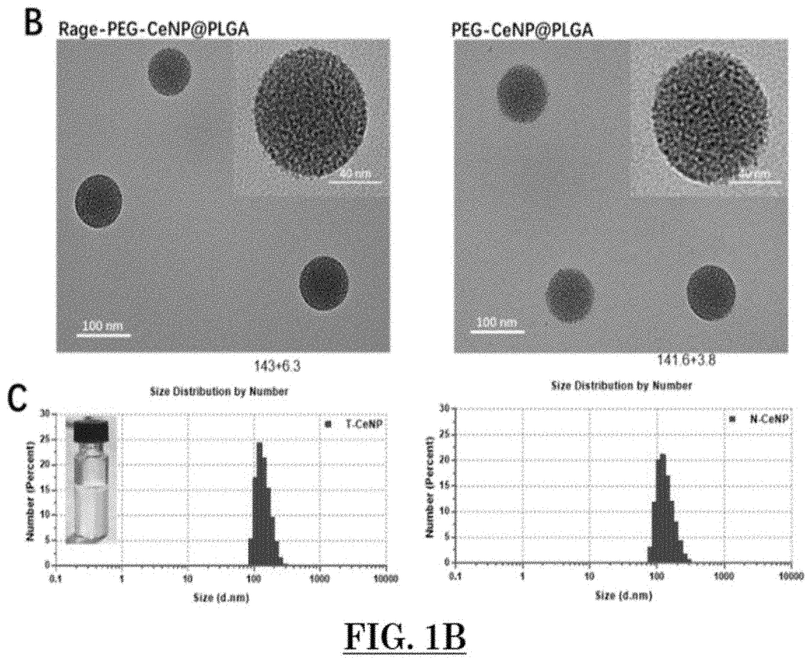

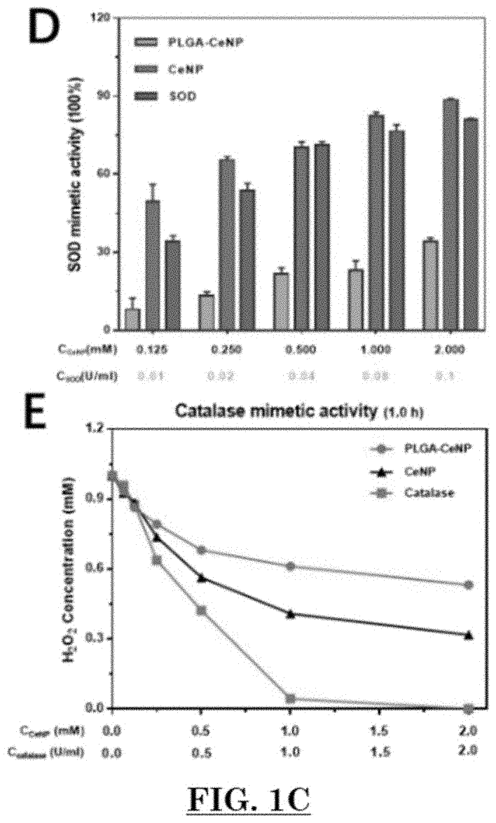

T-CeNPs were fabricated through emulsion method as shown in FIGS. 1A and 1B. The sizes of the T-CeNP and N-CeNP were around 140 nm as revealed by TEM (FIG. 1B) and DLS (FIG. 1C). FIG. 1C at d and e proved that, after being encapsulated in the PLGA matrix, the catalase and SOD mimicking catalytic activities of the CeNP were retained. FIGS. 1A, 1B, and 1C show characterization of T-CeNP at: (a) schematic fabrication of T-CeNP; (b) TEM images; (c) DLS size of T-CeNP and N-CeNP; evaluation of (d) catalase mimetic activity and (e) SOD mimetic activity of T-CeNP and free CeNP by referencing to catalase and SOD. Data were expressed as mean±s.d. (n=5).

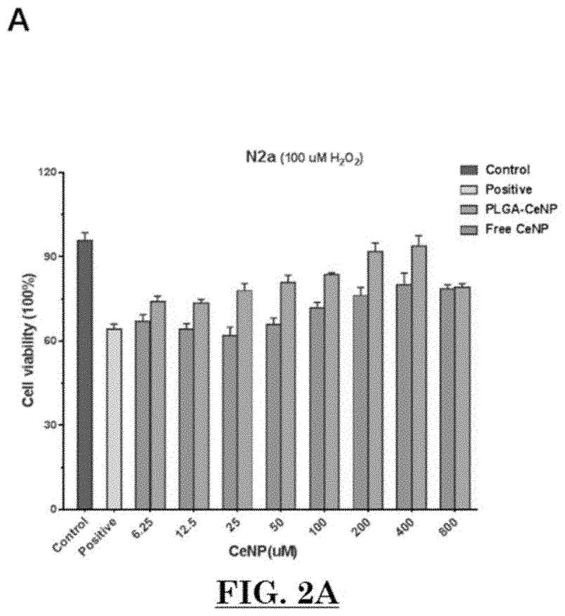

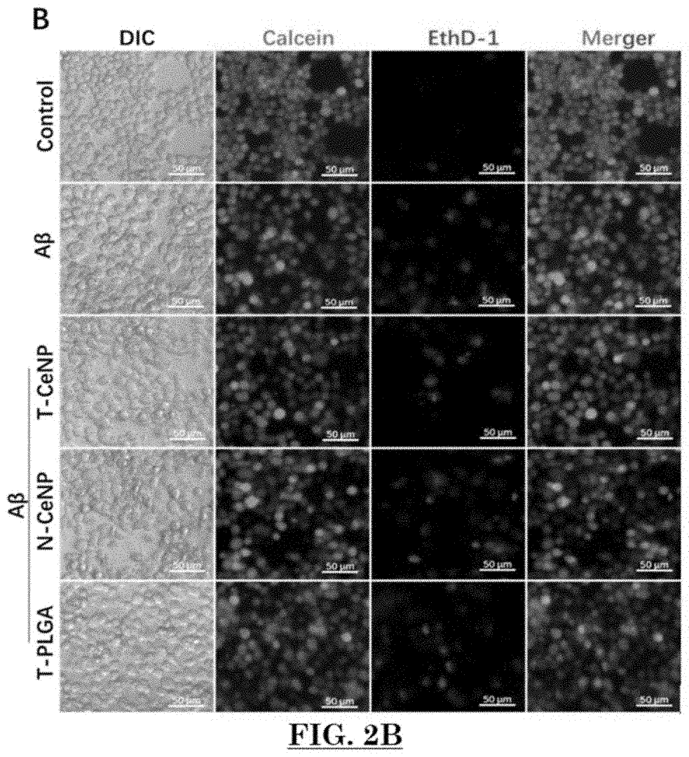

FIGS. 2A and 2B revealed that T-CeNP could rescue N2a cells from the damaging of H₂O₂ and Aβ. FIGS. 2A and 2B show the protective effect of T-CeNP on Aβ oligomers and H₂O₂ induced cytotoxicity to N2a cells at (a) cell viability of N2a cells measured by MTT assay. N2a cells were pre-treated with various nanop articles for 12 h, followed by incubating with 100 μM of H₂O₂ for another 24 h; at (b) cell viability of N2a cells measured by live/dead staining. N2a cells were pre-treated with 100 μM of various nanoparticles for 12 h, followed by incubating with 10 μM of Aβ oligomers for another 24 h. Fluorescence images of live (green) and dead (red) N2a cells co-stained with Calcein-AM and EthD-1 after undergoing the treatment. Data were expressed as mean±s.d. (n=5).

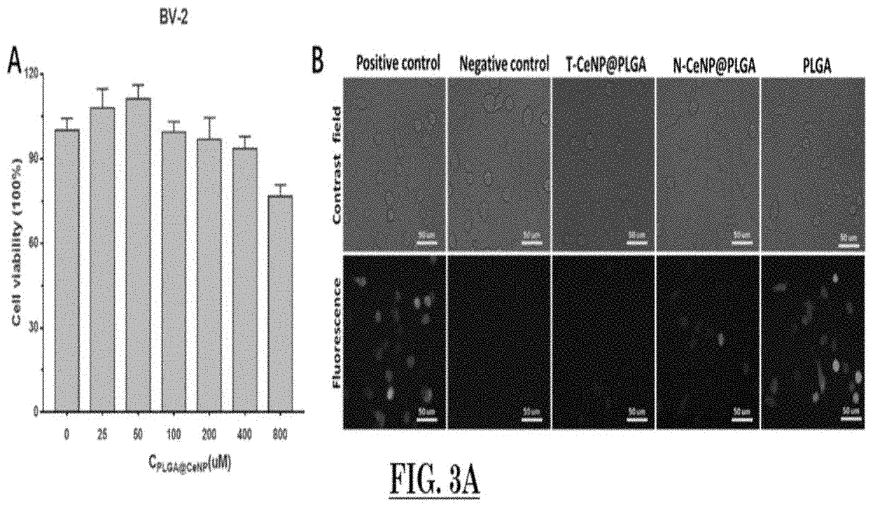

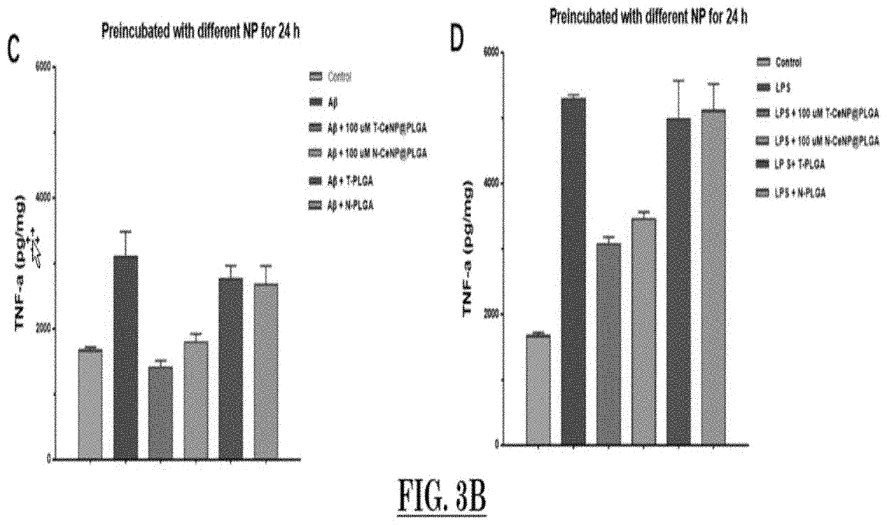

The T-CeNP is non-toxic for the BV-2 cells up to 400 μM (FIG. 3A). FIG. 3B indicated that T-CeNP is effective in attenuate the ROS induced by H₂O₂. Furthermore, FIG. 3B at c-d proved that T-CeNP could reduce the Aβ and LPS induced inflammatory response in BV-2 cells. FIGS. 3A and 3B show antioxidant and anti-inflammatory effects of T-CeNP in BV-2 cells at (a) cell viability of BV-2 cells measured by MTT assay after incubation with different concentration of T-CeNP for 24 h; at (b) fluorescence images of intracellular ROS in N2a cells. N2a cells were pre-treated with 100 μM of various nanoparticles for 12 h, followed by incubating with 100 μM of H₂O₂ for another 24 hours. The intracellular ROS level was determined by DCFH-DA. The quantitative analysis of secretive TNF-α in BV-2 by using TNF-α ELIAS kit after following treating with various nanoparticles. At (c) Aβ oligomers or at (d) LPS. Data were expressed as mean±s.d. n=5 for panel A and n=3 for panel C and D.

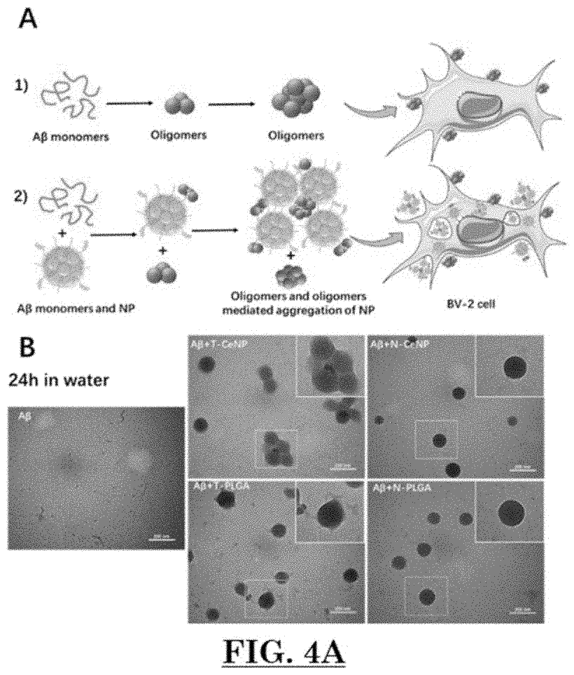

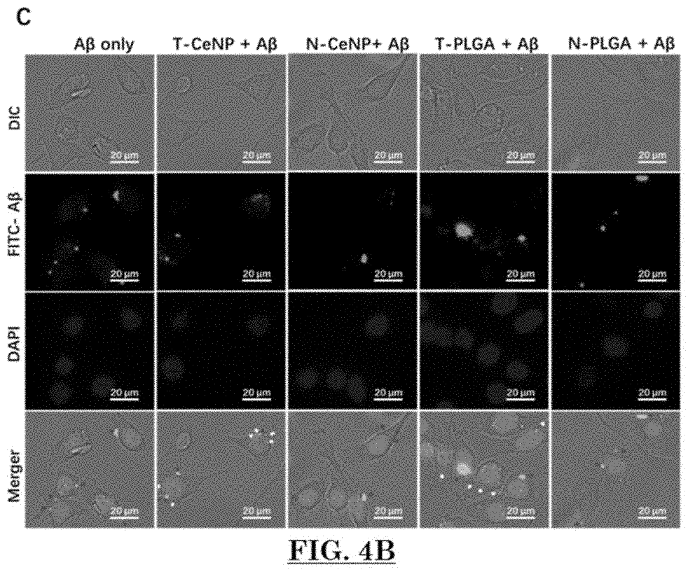

FIG. 4A found that T-CeNP could disrupt the formation of Aβ oligomers and facilitate the clearance of Aβ by BV-2 cells (see FIG. 4B). FIGS. 4A and 4B show T-CeNP mediated aggregation of Aβ oligomers and its uptake by BV-2 cells at (a) schematic formation of Aβ oligomers, nanocomposites of T-CeNP and Aβ oligomers and their internalization in BV-2 cells; at (b) TEM images of Aβ oligomers and nanocomposites of various nanoparticles and Aβ oligomers after 24 h of incubation; at (c) distribution of various Aβ oligomers in BV-2 cells. Aβ was labeled with FITC (green). The white arrows indicated intracellular Aβ oligomers and the red ones indicated Aβ oligomers adhered on cell surface.

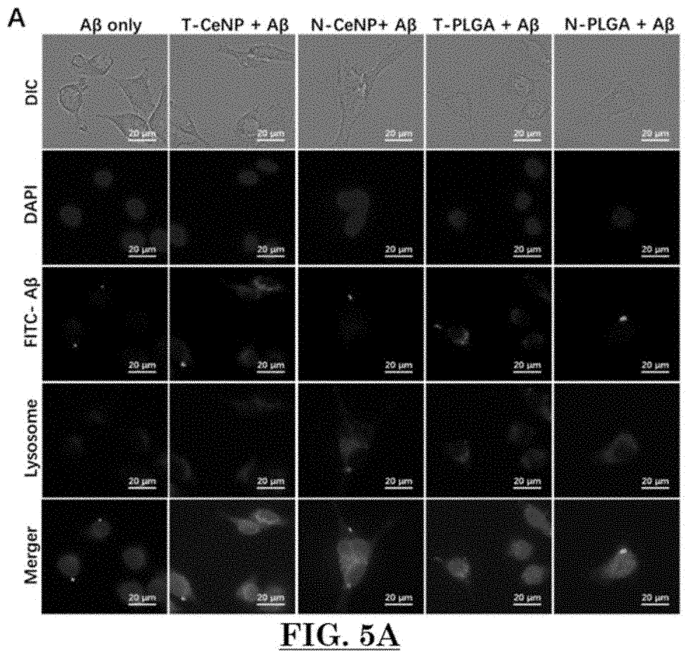

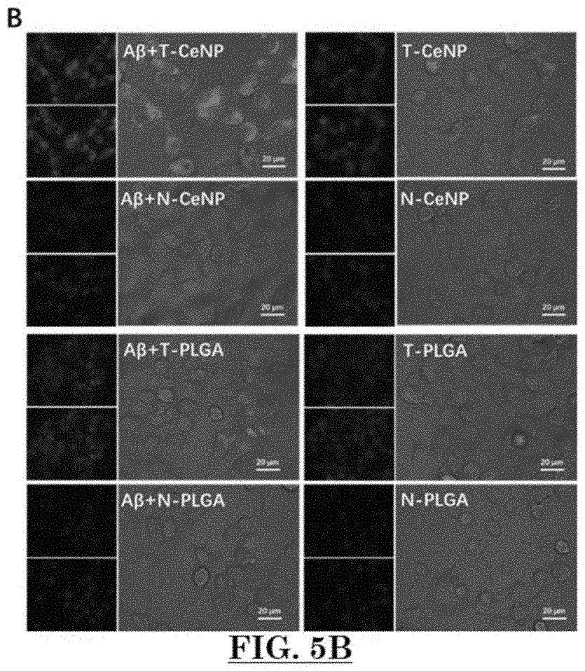

It was revealed that T-CeNP facilitates the cellular uptake of Aβ oligomers by BV-2 cells (FIG. 5A) and the existence of Aβ oligomers also promote the uptake of T-CeNP by BV-2 cells (FIG. 5B). FIGS. 5A and 5B show T-CeNP mediated Ab oligomers lysosomal location and AB mediated T-CeNP uptake by BV-2 at: (a) co-location of Aβ oligomers and lysosome in BV-2 cells. Aβ was labeled with FITC (green) and lysosome were stained with Lysotracker Red DND-99 (red). At (b) uptake of various nanoparticles and their aggregation with Aβ by BV-2 cells. All nanoparticles were labeled with Nile Red.

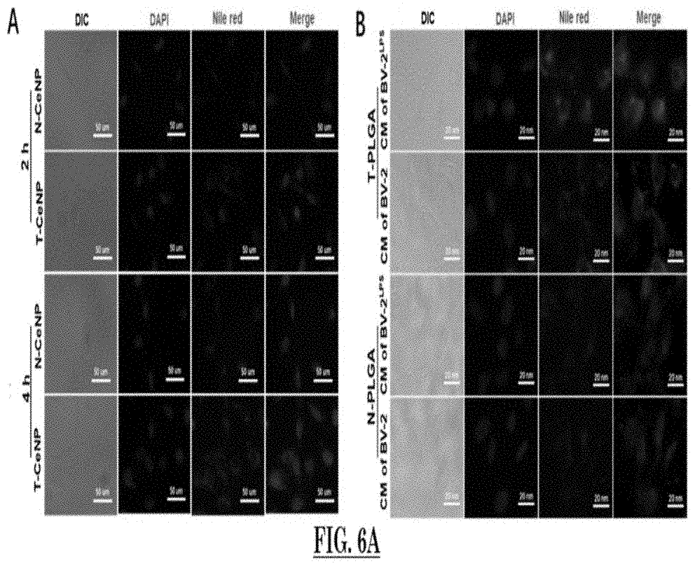

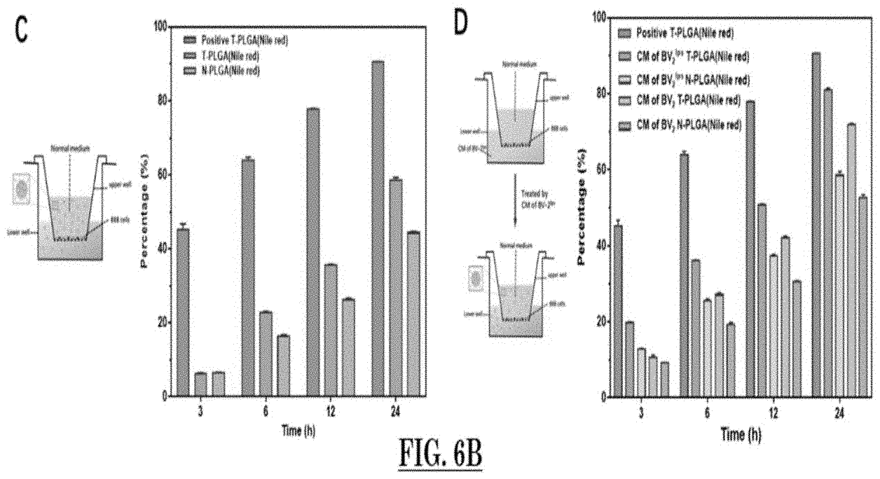

T-CeNP is more effective in entering the endothelium cells of BBB than the N-CeNP (FIG. 6A) and penetrating the in vitro BBB model (FIG. 6B). Furthermore, the conditioned medium from BV-2 cells treated with LPS facilitated the cellular uptake of T-CeNP (FIG. 6A) and promoted its BBB penetration (FIG. 6B). FIGS. 6A and 6B show cellular uptake in hCMEC/D3 cells and in vitro BBB crossing efficiency of nanoparticles at: (a) confocal laser scanning microscopy images of hCMEC/D3 cells incubated with different nanoparticles for 2 and 4 h; at (b) confocal laser scanning microscopy images of hCMEC/D3 cells pretreated with conditioned medium from BV-2 cells or LPS treated BV-2 cells, incubated with different nanoparticles for 4 h; at (c) a schematic of in vitro BBB model and quantitative analysis of penetration efficiency of various nanoparticles through the BBB model; at (d) a schematic of in vitro BBB model and quantitative analysis of the influence of pretreatment with conditioned medium from BV-2 cells or LPS treated BV-2 cells on the penetration efficiency of various nanoparticles through the BBB model. Data were expressed as mean±s.d. (n=3).

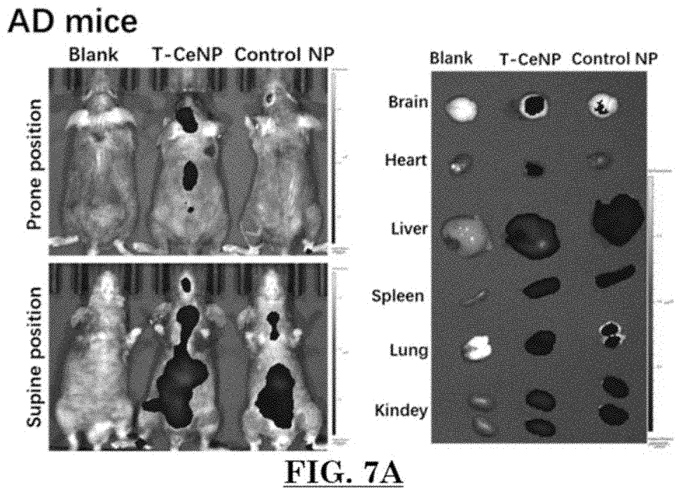

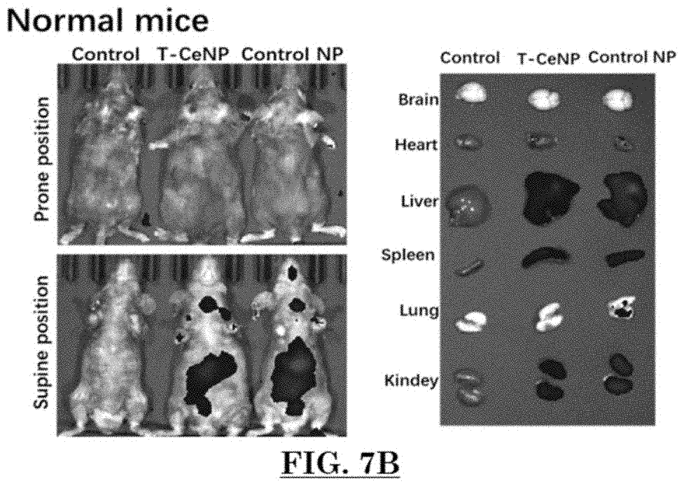

T-CeNP showed high targeting efficiency for the brain of AD mice, not in the brain of normal mice (FIG. 7). FIGS. 7A and 7B show T-CeNP distribution in AD mice and normal mice via in vivo and ex vivo fluorescence images of Cy5 labeled T-CeNP and N-CeNP distribution in (a) AD mice and (b) normal mice.

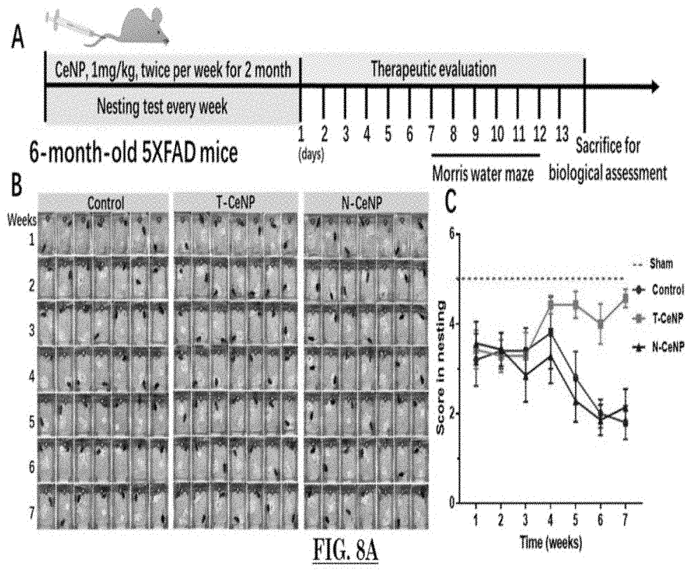

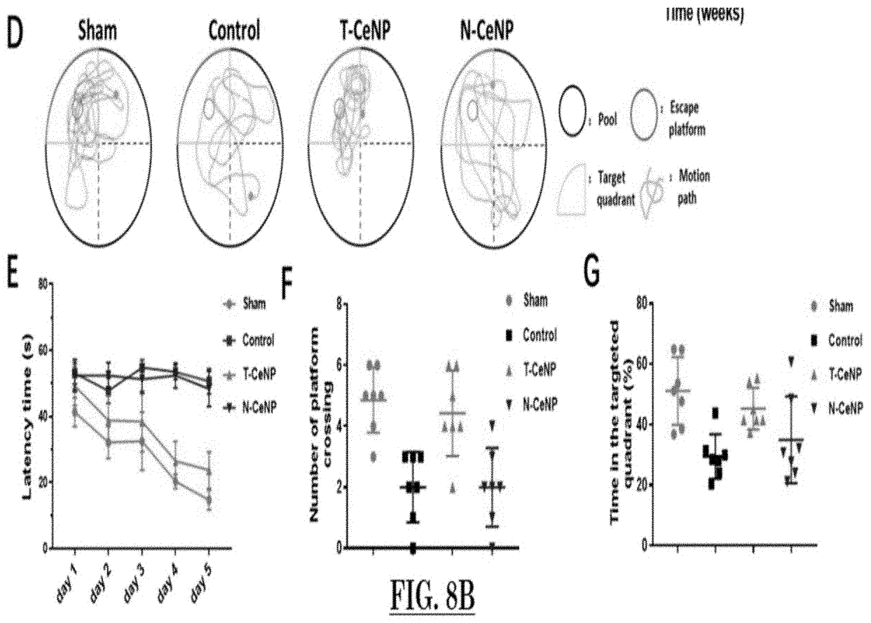

The animals received T-CeNP showed improved nesting capacity (FIG. 8A), enhanced spatial learning and memory abilities (FIG. 8B) compared with that in the control and non-targeted group. FIGS. 8A and 8B show Behavioral evaluation of T-CeNP therapy in 5XFAD mice; at (a) a time schedule of drug administration and therapeutic evaluation; (b) a photograph of nest-construction; (c) corresponding nest-constructing scores in different groups; (d) representative swimming path of mice in different groups in the Morris water maze test (MWM); (e) escape latency; (f) number of times crossing the escape platform used to locate; (g) percentage of time spent in the quadrant where the escape platform used to locate. Data were expressed as mean±s.d. (n=7).

The current disclosure provides in various aspects methods for preparation of a brain targeted cerium oxide nanop article (CeNP) and its application in treating central neuronal system diseases. In an alternative embodiment, a system is provided for preparation of a brain targeted cerium oxide nanop article (CeNP) and its application in treating central neuronal system diseases. In a still further embodiment, methods of treatment for central neuronal system diseases as described and shown herein.

In another embodiment, the disclosure provides a hybrid nanop article that may include a poly (lactic-co-glycolic acid) nano-matrix and a plurality of cerium oxide nanoparticles. Still yet, the plurality of cerium oxide nanoparticles may be encapsulated in the poly (lactic-co-glycolic acid) nano-matrix by hydrophobic interaction. Further again, the plurality of cerium oxide nanoparticle may range in size from 2-20 nanometers. Still yet, the poly (lactic-co-glycolic acid) nano-matrix may range in size from 50-300 nanometer. Moreover, the plurality of cerium oxide nanoparticles may exhibit antioxidant and anti-inflammatory effects. Again, the hybrid nanoparticle may include a targeting ligand conjugated to the hybrid nanoparticle. Further, the targeting ligand may be a ligand targeting a receptor for advanced glycation end products (RAGE). Even further, the hybrid nanop article may be used in an effective amount for treatment of central neuronal system diseases. Again still, the central neuronal diseases may comprise Alzheimer's disease, Multiple sclerosis, Brain tumor, glioblastoma, neuroblastoma, Parkinson's disease, Epilepsy, neonatal hypoxic-ischemic, stroke, Amyotrophic lateral sclerosis, Huntington's disease, Spinal cord injury, brain injury, post-traumatic stress disorder, and/or frontotemporal dementia.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for preparation of a targeted cerium oxide nanop article comprising:
preparing poly(lactide-co-glycolide)-b-poly(ethylene glycol)-maleimide (PLGA-PEG-Mal);
affixing at least one alkanethiol to at least one cerium oxide nanop article; and
constructing at least one nanocluster of T-CeNP via:
mixing PLGA and PLGA-PEG-Mal with the at least one cerium oxide nanoparticle; and
forming a solution of the above and adding at least one receptor for advanced glycation endproducts.

2. The method for preparation of a targeted cerium oxide nanoparticle of claim 1, further comprising wherein the at least one alkanethiol comprises 1-octanethiol.

3. The method for preparation of a targeted cerium oxide nanoparticle (CeNP) of claim 1, further comprising introducing an effective amount of the targeted cerium oxide nanop article to at least one neuronal cell for treatment of a central neuronal system disease.

4. The method of claim 3, wherein the central neuronal system disease comprises Alzheimer's disease, Multiple sclerosis, Brain tumor, glioblastoma, neuroblastoma, Parkinson's disease, Epilepsy, neonatal hypoxic-ischemic, stroke, Amyotrophic lateral sclerosis, Huntington's disease, Spinal cord injury, brain injury, post-traumatic stress disorder, and/or frontotemporal dementia.

5. The method for preparation of a targeted cerium oxide nanoparticle (CeNP) of claim 1, further comprising introducing an effective amount of the cerium oxide nanoparticle to at least one neuronal cell to provide antioxidant or anti-inflammatory effects to the at least one neuronal cell.

6. The method for preparation of a targeted cerium oxide nanoparticle (CeNP) of claim 1, further comprising wherein the targeted cerium oxide nanoparticle is introduced to a subject and penetrates the blood brain barrier of the subject.

7. The method for preparation of a targeted cerium oxide nanoparticle (CeNP) of claim 1, further comprising the at least one cerium oxide nanoparticle ranges in size from 2-10 nanometers.

8. The method for preparation of a targeted cerium oxide nanoparticle (CeNP) of claim 1, further comprising the Poly (lactic-co-glycolic acid) nano-matrix ranges in size from 50-300 nanometers.

9. A hybrid nanoparticle comprising a Poly (lactic-co-glycolic acid) nano-matrix and at least one cerium oxide nanoparticle.

10. The hybrid nanoparticle of claim 9, wherein the at least one cerium oxide nanoparticle is encapsulated in the Poly (lactic-co-glycolic acid) nano-matrix by hydrophobic interaction.

11. The hybrid nanoparticle of claim 9, wherein the at least one cerium oxide nanoparticle ranges in size from 2-10 nanometers.

12. The hybrid nanoparticle of claim 9, wherein the Poly (lactic-co-glycolic acid) nano-matrix ranges in size from 50-300 nanometers.

13. The hybrid nanoparticle of claim 9, wherein the at least one cerium oxide nanoparticle exhibits antioxidant and/or anti-inflammatory effects.

14. The hybrid nanop article of claim 9, further comprising a targeting ligand bonded to the hybrid nanoparticle.

15. The hybrid nanoparticle of claim 9 used in an effective amount for treatment of a central neuronal system disease.

16. The hybrid nanoparticle of claim 15, wherein the central neuronal disease comprises Alzheimer's disease, Multiple sclerosis, Brain tumor, glioblastoma, neuroblastoma, Parkinson's disease, Epilepsy, neonatal hypoxic-ischemic, stroke, Amyotrophic lateral sclerosis, Huntington's disease, Spinal cord injury, brain injury, post-traumatic stress disorder, and/or frontotemporal dementia.
